(19) United States
(12) Patent Application Publication
(10) Pub. No.: US 2019/0262612 A1
(43) Pub. Date: Aug. 29, 2019

(54) NEURAL CO-PROCESSOR FOR RESTORATION AND AUGMENTATION OF BRAIN FUNCTION AND ASSOCIATED SYSTEMS AND METHODS

(71) Applicant: University of Washington

(72) Inventors: Rajesh Rao, Seattle, WA (US); Eberhard Fetz, Seattle, WA (US)

(21) Appl. No.: 16/319,648

(22) PCT Filed: Jul. 26, 2017
(86) PCT No.: PCT/US17/44012

§ 371 Date: Jan. 22, 2019

Related U.S. Application Data
(60) Provisional application No. 62/366,951, filed on Jul. 26, 2016.

(51) Int. Cl. A61N 1/36; A61B 5/04; A61N 1/05; A61B 5/00; A61B 5/11

(52) U.S. Cl. CPC A61N 1/36103; A61N 1/36139; A61N 1/36031; A61B 5/1124; A61N 1/0529; A61N 1/0551; A61B 5/4005; A61B 5/04001

(57) ABSTRACT

Systems and methods for restoring or augmenting neural function by inducing new neural connections in a nervous system of a human patient or able-bodied individual are disclosed. One method for inducing new neural connections to restore lost neural function or augment neural function includes receiving neural signals from the nervous system of the individual and/or signals from an external sensor or information source. A stimulation pattern is generated based on (a) the neural signals and/or external information sources, and (b) a neural model, and the stimulation pattern is output to the nervous system of the individual. Stimulation of the nervous system based on the stimulation pattern computed by the neural model produces a measureable output by the individual. An error signal can be determined based at least in part on the measureable output and a desired output, and the neural model can be adjusted based on the error signal.

### CROSS-REFERENCE TO RELATED APPLICATION(S)

The present application claims priority to U.S. Provisional Application No. 62/366,951, filed Jul. 26, 2016, and titled NEURAL CO-PROCESSOR FOR RESTORATION AND AUGMENTATION OF BRAIN FUNCTION, which is incorporated herein by reference in its entirety.

### STATEMENT REGARDING FEDERALLY SPONSORED RESEARCH

This invention was made with government support under Grant No. EEC-1028725, awarded by the National Science Foundation, and Grant No. R01 NS012542, awarded by the National Institutes of Health. The government has certain rights in the invention.

### TECHNICAL FIELD

The present technology relates generally to restoring and augmenting brain function. In particular, some embodiments of the present technology include systems and methods for inducing neuroplasticity in the nervous system of a human patient or for enhancing the capabilities of the nervous system of an able-bodied individual.

### BACKGROUND

The human brain is one of the world's most powerful autonomous computers, but nevertheless remains a fragile organ that can be difficult to train and repair, and that is limited by the sensory inputs and processing capacity provided by the human body. The parallel functions of and similarities between the brain and typical silicon computers have prompted considerable interest in the field of brain-computer interfaces. Brain-computer interfaces may be able to address some of the limitations of the human brain as well as bolster our understanding of an organ with many functions and operations that remain to be understood. More specifically, the computational capabilities of biological neural networks and silicon computers are complementary. For example, human brains commonly transfer information bidirectionally with computers through normal sensory and motor channels. However, transferring information through direct recording of neural activity and electrical stimulation of brain sites is much more challenging. Nevertheless, recent advances in interface technologies, computing systems, and our understanding of the human brain have sparked new investigations into the potential of brain-computer interfaces that directly record and/or stimulate the brain.

### BRIEF DESCRIPTION OF THE DRAWINGS

FIG. 1 is a schematic diagram of a system for inducing new neural connections in a nervous system of a human subject configured in accordance with embodiments of the present technology.

FIG. 2 is a flow diagram of a process or method for restoring neural function in accordance with embodiments of the present technology.

FIG. 3 is a partially schematic diagram of a particular embodiment of the system of FIG. 1 for restoring neural function in accordance with embodiments of the present technology.

FIG. 4 is a flow diagram of a process or method for augmenting neural function in accordance with embodiments of the present technology.

FIG. 5 is a partially schematic diagram of a particular embodiment of the system of FIG. 1 for augmenting neural function in accordance with embodiments of the present technology.

### DETAILED DESCRIPTION

Aspects of the present disclosure are directed generally toward systems for restoring or enhancing neural function by inducing new neural connections in a nervous system (e.g., in the brain or spinal cord of a human subject) and associated methods. In several of the embodiments described below, a system for inducing new neural connections in a nervous system of a human subject includes at least one sensor implanted in the subject for detecting and recording neural signals from a first region of the nervous system of the subject. In other embodiments, in addition to or instead of sensors implanted in the human body, the sensor may be positioned external to the body of the subject and configured to detect and record external signals. The sensors can be communicatively coupled to a computing device that receives the signals recorded by the sensors and generates a stimulation pattern based on (a) the received signals and (b) an adaptive artificial neural model. The system can further include a stimulating component implanted in and/or worn externally by the subject and communicatively coupled to the computing device for receiving the stimulation pattern. The stimulating component can stimulate a second region of the nervous system of the subject based on the stimulation pattern to generate a measurable response (e.g., one or more neural patterns or signals internal to the nervous system, motor behavior of the subject, sensory behavior of the subject, etc.). An error signal can be determined by comparing the measurable response to a desired response (e.g., from rehabilitation procedures), and the artificial neural model can be adjusted based on the error signal and/or based on data, information, or signals other than the error signal.

In the following detailed description, specific details are set forth to provide an understanding of the present technology. However, the present technology may be practiced without some of these specific details. In some instances, well-known structures and techniques have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms may even be emphasized below; however, any terminology intended to be interpreted in any restricted manner will be overtly and specifically defined as such in this Detailed Description section.

The present technology can include a computing device (a “co-processor”) that is effectively arranged “in parallel” with a portion of the nervous system of the subject (e.g., the brain) and that operates synergistically with the brain to jointly optimize a task or behavior. The systems and methods described herein can be used to augment the natural computational abilities of the brain and promote neural plasticity for the creation of new natural pathways between different areas of the brain, nervous system, or body organs. These new neural pathways can function to replace lost neural function (e.g., resulting from injury) or to augment existing neural function in the brain or other parts of the nervous system.

### Suitable System(s)

The following discussion provides a general description of a suitable environment in which the present technology may be implemented. Although not required, aspects of the technology are described in the general context of computer-executable instructions, such as routines executed by a general-purpose computer. Aspects of the technology can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, and/or a short-range radio network (e.g., via Bluetooth)). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g. a Bluetooth network) on a propagated signal on a propagation medium (e.g., an electromagnetic wave(s) or a sound wave) over a period of time, or may be provided on any analog or digital network (e.g., packet switched, circuit switched, or other scheme).

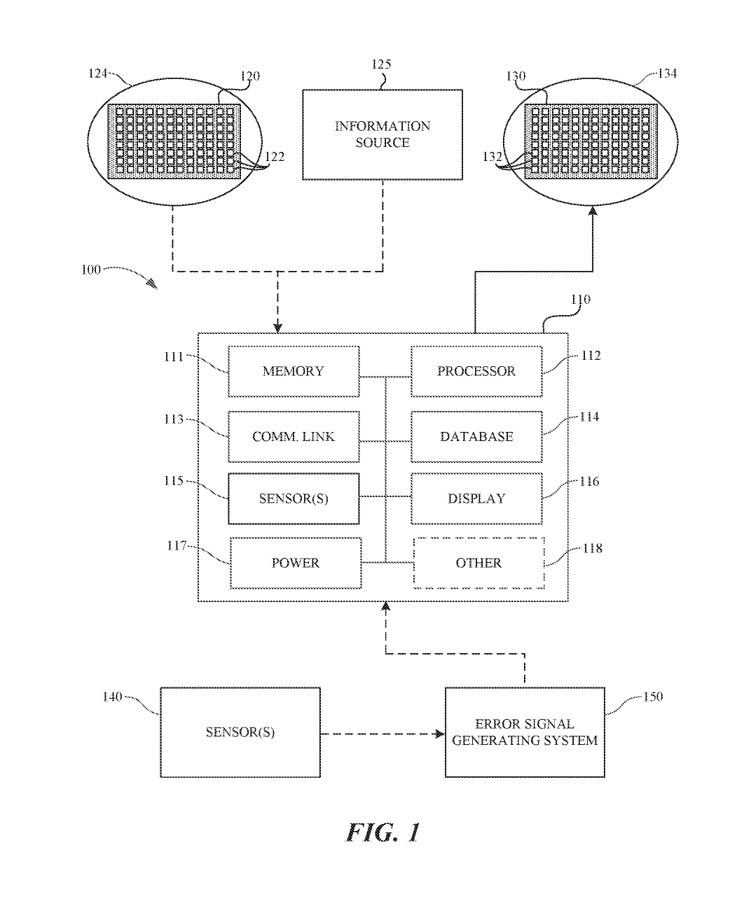

FIG. 1 is a schematic diagram of a system 100 for augmenting or restoring neural function by inducing new neural connections in a nervous system of a subject (e.g., a human patient or able-bodied individual) configured in accordance with an embodiment of the present technology. The system 100 includes at least a processing subsystem 110, a sensor module 120 and/or information source 125 communicatively coupled to the processing subsystem 110, and a stimulation module 130 communicatively coupled to the processing subsystem 110. A simplified example of a suitable processing subsystem is described in “The Neurochip-2: An Autonomous Head-Fixed Computer for Recording and Stimulating in Freely Behaving Monkeys,” by Stavros Zanos, Andrew G. Richardson, Larry Shupe, Frank P. Miles, and Eberhard E. Fetz, IEEE Transactions on Neural Systems And Rehabilitation Engineering, Vol. 19, No. 4, pg. 427-435, August 2011, which is incorporated herein by reference in its entirety. An application of such a subsystem to induce neuroplasticity using a simple spike detection method is described in “Long-term motor cortex plasticity induced by an electronic neural implant,” by Andrew Jackson, Jaideep Mavoori, and Eberhard E. Fetz. Nature. Vol. 444, pg. 56-60, Nov. 2, 2006, which is also incorporated herein by reference in its entirety.

The sensor module 120 can be any type of sensor such as, for example, a sensor configured to detect neural signals in the nervous system of the subject (e.g., for detecting neuroelectrical activity, neurochemical activity, etc.), an ultrasonic sensor, an infrared sensor, or another type of sensor. Accordingly, the sensor module 120 can be configured to detect signals that are external (e.g., infrared signals from an environment around the subject) and/or internal (e.g., neural signals from the nervous system of the subject) relative to the subject. In some embodiments, the sensor module 120 is implanted proximate a region of interest 124 in the subject. The region of interest 124 can be, for example, a region of the subject's brain or another portion of the subject's nervous system. In some embodiments, the sensor module 120 can be a multi-channel sensor that includes a plurality of individual sensor elements 122. Each sensor element 122 can be configured to detect one or more signals generated or detected at a different location in and/or on the region of interest 124. For example, where the sensor module 120 is implanted within and/or worn externally by the subject and the region of interest 124 is a region of the subject's brain, the sensor elements 122 can be configured to provide a spatial and/or spatiotemporal sampling of neural signals generated over the region of interest 124. In some embodiments, the number of individual sensor elements 122 (e.g., measurement channels) in the sensor module 120 can be selected to correspond to a characteristic of the region of interest 124 (e.g., the physical structure of the region, the degrees of freedom in a detectable signal at the region, the numbers of neurons in the region, etc.).

In some embodiments, the information source 125 is provided instead of or in addition to the sensor module 120. The information source 125 can comprise any source of information such as the Internet, a data structure containing data, a source of user input, another nervous system, etc., for providing input to the processing subsystem 110.

The stimulation module 130 is positioned at or proximate to a stimulation region 134 in the subject (e.g., implanted in and/or worn externally by the subject). The stimulation region 134 can be, for example, a region of the brain of the subject, another region of the nervous system of the subject (e.g., the spinal cord or different nerves), a region of muscle, and/or a region of an organ of the subject. The stimulation module 130 can include any invasive or non-invasive hardware for stimulating the stimulation region 134 of the subject. For example, the stimulation module 130 can be configured to stimulate the stimulation region 134 using one or more of: electrical activation, optical activation, magnetic activation, ultrasonic activation, and chemical activation. In some embodiments, the stimulation module 130 can be a multi-channel module that includes a plurality of individual stimulating elements 132. Each stimulating element 132 can be configured to stimulate the stimulation region 134 at a different location in and/or on the stimulation region 134. For example, the stimulation region 134 can be a region in the subject's brain (e.g., the primary motor cortex of the brain, a region of the brain implicated in a certain neural function, etc.) and the stimulating elements 132 can be configured to provide a spatially and/or spatiotemporally differing stimulation pattern along and/or through the stimulation region 134. In some embodiments, the number of individual stimulating elements 132 (e.g., stimulation channels) in the stimulation module 130 can be selected to correspond to a characteristic (e.g., the physical structure) of the stimulation region 134.

The processing subsystem 110 comprises several components including memory 111 (e.g., one or more computer readable storage modules, components, devices, etc.) and one or more processors 112. The memory 111 can be configured to store information (e.g., signal data, subject information or profiles, environmental data, data collected from one or more sensors, media files, etc.) and/or executable instructions that can be executed by the one or more processors 112. The memory 111 can include, for example, instructions for processing multi-channel sensor data from the sensor module 120 and/or information from the information source 125 using an adaptive method such as an artificial neural model, for suppressing noise and other artifacts from signals received from the sensor module 120, for generating stimulation patterns for output to the stimulation module 130 based on the adaptive method, and for adjusting a neural model or other adaptive method, as described in further detail below.

The processing subsystem 110 also includes communication components 113 (e.g., a wired communication link and/or a wireless communication link (e.g., Bluetooth, Wi-Fi, infrared, and/or another wireless radio transmission network)) and a database 114 configured to store data (e.g., signal data acquired from the region of interest, equations, filters, etc.) used in the techniques for co-adapting with and inducing new neural connections in the subject, as disclosed herein. One or more first sensors 115 can provide additional data for use in inducing new neural connections in the subject. The sensors can also provide other data pertinent to a condition and/or environment of the subject. For example, the one or more first sensors 115 may include one or more ECoG sensors, voltammetry sensors of neurochemicals, blood pressure monitors, galvanometers, accelerometers, thermometers, hygrometers, blood pressure sensors, altimeters, gyroscopes, magnetometers, proximity sensors, barometers, etc. The first sensors 115 can also be configured to provide information about the system 100 itself, such as an operating condition (e.g., power level, noise level, etc.) of any or all of the components included therein. One or more displays 116 can provide video output and/or graphical representations of data obtained by the system 100, and can include one or more devices (e.g., keyboards, mice, joysticks, number pads, etc.) for supplying a user input at the displays 116. A power supply 117 (e.g., a power cable, one or more batteries, and/or one more capacitors) can provide electrical power to components of the processing subsystem 110 and/or the system 100. In embodiments that include one or more batteries (e.g., where the system 100) is a portable system), the power supply 117 can be configured to recharge, for example, via a power cable, inductive charging, and/or another suitable recharging method. Furthermore, in some embodiments, the processing subsystem 110 may include one or more additional components 118 (e.g., one or more microphones, cameras, Global Positioning System (GPS) sensors. Near Field Communication (NFC) sensors, etc.).

In some embodiments, the processing subsystem 110 can include one or more components partially or wholly incorporated into the sensor module 120, information source 125, and/or stimulation module 130. In other embodiments, however, the processing subsystem 110 may include components remote from the sensor module 120, information source 125, and/or stimulation module 130, and connected thereto by a communication network (e.g., the Internet and/or another network or cloud computers). In some embodiments, for example, at least a portion of the processing subsystem 110 may reside on a mobile device (e.g., a mobile phone, a tablet, a personal digital assistant, etc.) and/or a computer (e.g., a desktop computer, a laptop, etc.) communicatively coupled to the sensor module 120, information source 125, and/or stimulation module 130. Moreover, the processing subsystem 110 can be configured to be worn by the subject (e.g., carried by their body) and/or implanted in their body (e.g., proximate a region of their nervous system).

In some embodiments, the system 100 can optionally include one or more additional second sensors 140 and/or an error signal generating system 150. The second sensors 140 can be communicatively coupled to the error signal generating system 150 and/or directly communicatively coupled to the processing subsystem 110. The second sensors 140 can be configured to detect and/or record a measurable output by the subject resulting from stimulation of the stimulation region 134 by the stimulation module 130. The measurable output may be internal (e.g., a neural activity pattern, a neurochemical response, etc.) or external (e.g., a motor response) relative to the subject. Accordingly, the second sensors 140 may be internal (e.g., implanted within) relative to the subject and/or external to the subject. The optional error signal generating system 150 can be communicatively coupled to the processing subsystem 110 and configured to generate an error signal, as described in further detail below. The error signal generating system 150 can have some components generally similar to those of the system 10X) (e.g., one or more processors, sensors, memory, etc.). In some embodiments, the error signal generating system 150 stores or generates desired outputs corresponding to the measurable outputs recorded by the second sensors 140, compares the desired outputs to the measurable outputs, and calculates an error signal.

In certain embodiments, the second sensors 140 and/or the error signal generating system 150 may be partially or wholly incorporated into the processing subsystem 110, the stimulation module 130, the information source 125, and/or the sensor module 120, or omitted entirely. For example, the function of the second sensors 140 may be performed by the first sensors 115 of the processing subsystem 110, or the second sensors 140 can be omitted and any information about the measurable output from the subject can be determined by other means (e.g., by an observing physician, researcher, user, etc.) and directly input via the processing subsystem 110 (e.g., at the display 116).

### Suitable Methods

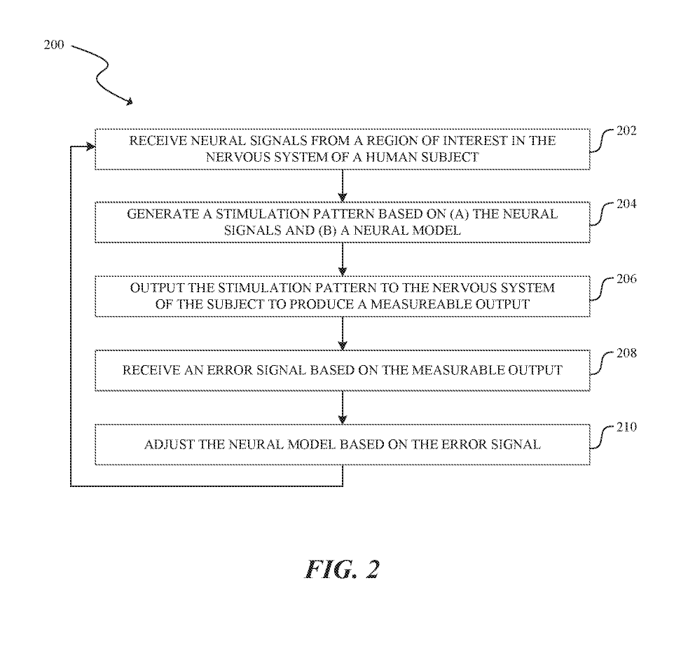

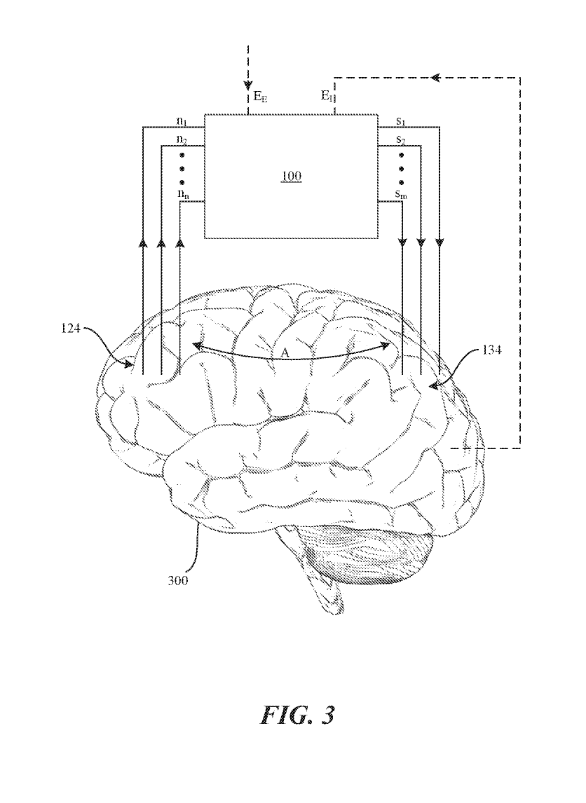

FIG. 2 is a flow diagram of a process 200 for restoring lost neural function in a subject (e.g., a human patient) in accordance with an embodiment of the present technology. The lost neural function can be the result of, for example, a stroke or spinal cord injury of the subject. The process 200 can include instructions stored, for example, in the memory 111 of the system 100 (FIG. 1) that are executable by the one or more processors 112 (FIG. 1). In some embodiments, portions of the process 200 are performed by one or more hardware components (e.g., the sensor module 120 and/or stimulation module 130 of FIG. 1). In certain embodiments, portions of the process 200 are performed by a device external to the system 100 of FIG. 1. FIG. 3 is a schematic illustration of a particular embodiment of the system 100 for restoring lost neural function in, for example, a brain 300 of a human subject. For sake of illustration, some features of the process 200 will be described in the context of the embodiment shown in FIG. 3.

Beginning at block 202, the process 200 comprises receiving neural signals from a region of interest in a nervous system of a subject. For example, in the embodiment illustrated in FIG. 3, the system 100 receives a plurality of neural signals n₁-nₙ (e.g., via the sensor module 120) from the region of interest 124, which is a region of the brain 300 of the subject. The neural signals n₁-nₙ are representative of neuroelectrical and/or neurochemical signals generated in the brain 300. In some embodiments, each signal n₁-nₙ is detected at and/or recorded from a different spatial location of the region of interest 124, and/or at one or more different times. In some embodiments, the neural signals n₁-nₙ are recorded while the subject performs a particular task. For example, if the goal of the process 200 is to restore a motor function of the subject, the neural signals n₁-nₙ may be recorded while the subject performs or attempts to perform the specific motor function to be restored. In other embodiments, if the goal of the process 200 is to restore or augment a cognitive function of the subject, the neural signals n₁-nₙ may be generated by the subject to access the function or information on processing subsystem 110 or the information source 125.

At block 204, the process generates a stimulation pattern based on the neural signals received at block 202, and based on a neural model. The memory 111 (FIG. 1) of the system 100 can store instructions executable by the one or more processors 112 (FIG. 1) for implementing the neural model. The neural model can include one or more algorithms for mapping inputs (e.g., the neural signals n₁-nₙ) to outputs (e.g., the stimulation pattern). For example, the neural model can be a computational learning algorithm or machine learning method for function approximation, such as a parametric or nonparametric regression method, or a network of biologically-inspired units (e.g., a neural network). Suitable parametric and nonparametric regression methods further include, but are not limited to: generalized linear models (GLMs), logistic and polynomial regression, Gaussian process regression, and other Bayesian methods. Biologically-inspired network learning models include, but are not limited to, networks using standard weighted-sum-with-nonlinearity type neurons, and more sophisticated neuronal models based on integrate-and-fire or biophysically realistic Hodgkin-Huxley type neurons. Regardless of the specific neural model, and as described in greater detail below, the parameters of the neural model can be adapted to map the input signals to an appropriate stimulation pattern.

At block 206, the process outputs the generated stimulation pattern to the nervous system of the subject to produce a measureable output within or outside the nervous system of the subject. For example, as illustrated in the embodiment of FIG. 3, the stimulation pattern can be output to the nervous system at the stimulation region 134, which can be a region of the brain 300 of the subject. In other embodiments, the stimulation region 134 can be a different region of the brain, a different region in the nervous system (e.g., a region of the spinal cord), and/or a region of muscle or organ. In some embodiments, the stimulation region 134 is a different region than the region of interest 124. For example, the stimulation region 134 and region of interest 124 can be regions of the nervous system that are separated by an injured region. The injured region could be the result of a spinal cord injury, in which case the region of interest 124 can be a motor intention-forming region of the brain and the stimulation region 134 can be a region of the spinal cord below the injury. Similarly, the injured region could be caused by a stroke, traumatic brain injury, or disease, and can partially or fully disconnect the region of interest 124 from the stimulation region 134. The system 100 can therefore “bridge” or “straddle” an injured region of the nervous system and, as described in greater detail below, promote neural plasticity (e.g., increased neural connections) through the injured region and/or between the region of interest 124 and the stimulation region 134. In some embodiments (e.g., for sensory restoration), the system 100 may connect an external sensor (e.g., the sensor module 120) to an arbitrary brain region, or a brain region implicated in the sense to be restored.

The stimulation pattern can, for example, comprise a plurality of stimulation signals s₁-sₘ each configured to stimulate a different portion of the stimulation region 134 (e.g., via the individual stimulating elements 132 of the stimulation module 130 of FIG. 1). In some embodiments, the stimulation signals s₁-sₘ are operable to stimulate the stimulation region 134 simultaneously or nearly simultaneously, while in other embodiments the stimulation signals s₁-sₘ are operable to stimulate the stimulation region 134 over a varying time period. In certain embodiments, the (input) neural signals n₁-nₙ are directly mapped to the (output) stimulation signals s₁-sₘ such that there is a one-to-one correspondence between the signals (e.g., n=m). However, the mapping of the neural signals n₁-nₙ to the stimulation signals s₁-sₘ can be any suitable mapping (e.g., n/m). For example, the number of output stimulation signals s₁-sₘ can depend on the specific anatomy of the stimulation region 134 as well as the configuration of the sensor module 120 (e.g., the number of individual sensor elements 122.) The stimulation signals s₁-sₘ can include electrical signals, optical signals, magnetic signals, ultrasonic signals, voltammetric signals, and/or other suitable signals.

The measureable output produced by stimulation of the nervous system can be either external or internal to the subject. For example, external measureable outputs include behavioral or motor responses of the subject (e.g., a physical movement of the subject) and/or sensory responses of the subject (e.g., an ability to perceive a specific stimuli), while internal measureable outputs include neural responses of the subject (e.g., a specific neuroelectrical or neurochemical activity pattern detected in the brain). As described above, in some embodiments, the measurable output can be detected, recorded, and/or measured by one or more sensors (e.g., the second sensors 140) of the system 100. In other embodiments, the measurable output is detected, recorded, and/or measured by another system, device, or person apart from the system 100 (e.g., a physician or researcher working with the subject).

At block 208, the process receives one or more error signals based, at least in part, on the measurable output. The error signals may be either internal or external to the subject depending on the measurable output. For example, as illustrated in FIG. 3, the system 100X) can receive either or both of an external error signal E_{E} and an internal error signal E_{I} from, for example, the error signal generating system 150 (FIG. 1). In some embodiments, the error signal is based on a comparison (e.g., a difference between) the measureable output and a desired output (e.g., a sensory or motor goal during rehabilitation therapy). The error signal generating system 150 can store and/or generate the desired outputs and compare the desired outputs to the measurable outputs to produce the error signals E_{E} and/or E_{I}. For example, where the measurable output is an external motor response (e.g., a hand movement), the error signal E_{E} can be based at least in part on a difference (e.g., a physical distance) between the hand movement of the subject and a desired hand movement of the subject. Similarly, where the measureable output is a specific neural activity pattern, the error signal E_{I} can be based on the difference between the specific neural activity pattern and a desired neural activity pattern. In some embodiments, the desired neural activity pattern can be pre-determined (i.e., known) from a previously-generated mapping of neural activity patterns to specific sensory or motor responses of the subject. If the desired neural activity pattern is not known in advance, the error signal generating system 150 can be configured to map recorded neural activity patterns (i.e., those generated by stimulation of the nervous system of the subject) to certain responses of the subject (e.g., hand movements or other motor, behavioral, or sensory responses) to determine and/or optimize the desired neural activity patterns.

In other embodiments, the error signal E_{I} can be an internal reward-based signal (e.g., a neurochemical reward prediction error) measured in one or more of the reward centers of the brain of the subject. That is, stimulation of the brain of the subject can produce a measurable output in the form of a release of dopamine or other neurotransmitter corresponding to a “reward or reward prediction error.” In some embodiments, the error signal generating system 150 includes one or more sensors (e.g., the second sensors 140) for measuring such reward-based error signals E_{I} in reward centers of the brain 300 of the subject. The detection of a reward-based signal (e.g., the release of dopamine) can indicate a desired output resulting from the stimulation, and can be used to reinforce the behavior that produced the reward-based signal.

At block 210, the process adjusts the neural model based, at least in part, on the error signal. For example, the external or internal error signal can be used to adapt parameters (e.g., to adjust one or more weights or values of one or more variables defining the neural model) of the neural model using an optimization method for minimizing error. In some embodiments, where the error signal is a reward-based error signal, the parameters of the neural model can be adapted to maximize the reward-based signal. The parameters of the neural model can be adapted offline, or adapted online in response to streaming input of the error signal (e.g., from the error signal generating system 150). In some embodiments, the parameters of the neural model can be adjusted based on user input, data, information, and/or signals other than the error signal, either continuously or at certain offline times. That is, in certain embodiments the process 200 is an adaptive process for restoring lost neural function in which the adjustments to the neural model are based on signals or information different than an error signal.

Lastly, the system returns to block 202 and the process 200 can be repeated. The neural model can therefore be optimized through iterative adjustments to minimize error between a resulting output and a desired output while, at the same time, inducing new neural connections between the region of interest 124 and the stimulation region 134 (represented by arrow A in FIG. 3). The new neural connections may increase plasticity through and/or around an injured region of the nervous system that is between these regions to restore function that was previously lost as a result of an injury (e.g., a stroke or spinal cord injury). Thus, the process 200 causes the system 100 to “co-adapt” and jointly optimize with the nervous system of the subject to promote neuroplasticity, rewiring, and creation of new neural pathways between populations of neurons for restoration and rehabilitation. Over time, dependence on the system 100 may be gradually reduced and the system 100 may be removed if sufficient function has been restored by the neuroplasticity induced by the use of system 100 over an extended period of time. In some embodiments, the system 100 may be configured as a permanent neural prosthesis.

In contrast to conventional brain-computer interfaces that use neural activity in the brain to control external devices, the present technology is directed to a bidirectional brain-computer interface having recurrent connections that allow activity-dependent stimulation of the brain, spinal cord, or muscles. Such a brain-computer interface can be made sufficiently small to be worn externally and/or implanted in a subject and operate autonomously during hours of free behavior.

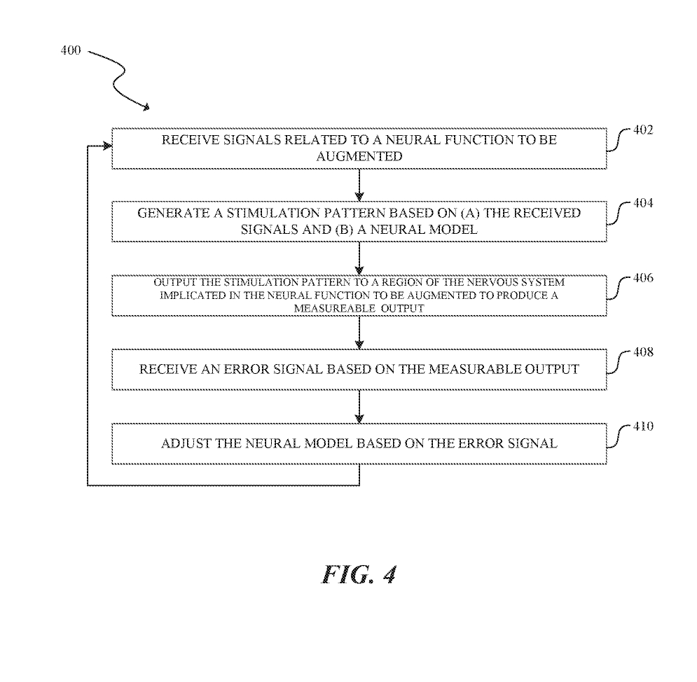

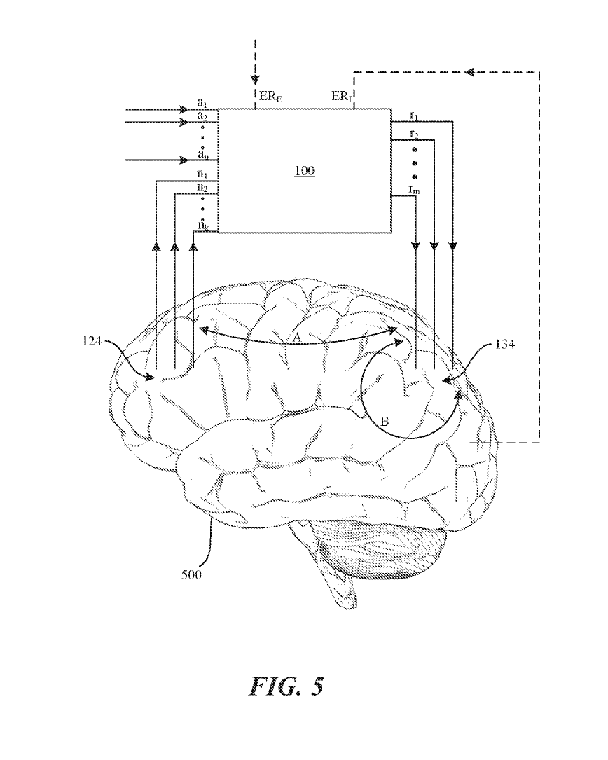

FIG. 4 is a flow diagram of a process 400 for augmenting neural function in a human subject (e.g., an able-bodied individual) in accordance with an embodiment of the present technology. The process 400 can include instructions stored, for example, in the memory 111 of the system 100 (FIG. 1) that are executable by the one or more processors 112 (FIG. 1). In some embodiments, portions of the process 400 are performed by one or more hardware components (e.g., the sensor module 120 and/or stimulation module 130 of FIG. 1). In certain embodiments, portions of the process 400) are performed by a device external to the system 100 of FIG. 1. FIG. 5 is a schematic illustration of a particular embodiment of the system 100 for augmenting neural function in, for example, a brain 500 of a human subject. For the sake of illustration, some features of the process 400 will be described in the context of the embodiment shown in FIG. 5.

Beginning at block 402, the process 400 includes receiving augmentation signals related to a neural function to be augmented. For example, in the embodiment illustrated in FIG. 5, the system 100 receives a plurality of augmentation signals a₁-aₙ from, for example, the sensor module 120 or information source 125 of FIG. 1. The process further includes receiving neural signals (e.g., via the sensor module 120) from a region of interest of the nervous system of the subject. For example, as shown in FIG. 5, the system 100 receives a plurality of neural signals n₁-nₖ from the region of interest 124, which is a region of the brain 500 of the subject. In some embodiments, the sensor module 120 includes an artificial sensor (e.g., infrared or ultrasonic sensor) such that the augmentation signals a₁-aₙ correspond to received artificial signals (e.g., infrared or ultrasonic signals). In other embodiments, the augmentation signals a₁-aₙ can relate to any function to be augmented and can be received from an information source such as the Internet. The augmentation signals a₁-aₙ are processed in combination with the internally recorded neural signals n₁-nₖ to achieve augmentation of neural function in the presence of ongoing neural activity in the brain of the subject.

In yet other embodiments, the augmentation signals a₁-aₙ are not derived from external sensors but are the neural signals n₁-nₖ themselves received from a region of interest (e.g., the region of interest 124) in the nervous system of the subject. For example, the augmentation signals a₁-aₙ can be neural signals detected and/or recorded at the prefrontal cortex of the brain 500 of the subject with the goal of augmenting motor skill-learning, short-term memory, or planning capabilities of the subject. In such embodiments where the augmentation signals a₁-aₙ are internal to the subject, the embodiment illustrated in FIG. 3 may be used for augmentation. That is, the neural signals n₁-nₙ can be received for augmenting or accelerating certain neural functions (e.g., certain types of memory, planning, perception, and motor skill learning) in, for example, able-bodied individuals rather than restoring neural function in, for example, patients. The neural signals n₁-nₙ can also be volitionally controlled by the subject to access information or computations provided by the system 100.

At block 404, the process generates a stimulation pattern based on (a) the augmentation signals received at block 402, (b) the recorded neural signals, and (c) an adaptive artificial neural model. In some embodiments, the stimulation pattern is based only on the recorded augmentation signals. The neural model can be generally similar to the neural model described above with reference to FIG. 2. At block 406, the process outputs the stimulation pattern to a region of the brain or nervous system of the subject whose neural function is to be augmented (e.g., the stimulation region 134). For example, where the augmentation signals a₁-aₙ are received from the prefrontal cortex of the brain 500 for augmenting or accelerating motor skill learning, the stimulation region 134 may be a region of the primary motor cortex-a region of the brain implicated in motor skill learning. In other embodiments, the stimulation region 134 may be one or more arbitrary regions of the brain 500 (e.g., when it is not known which single region of the brain might be implicated in the function to be augmented).

As illustrated in the embodiment of FIG. 5, the stimulation pattern can, for example, comprise a plurality of stimulation signals r₁-rₘ (e.g., electrical signals, optical signals, magnetic signals, ultrasonic signals, and/or neurochemical signals) each configured to stimulate a different portion of the stimulation region 134 (e.g., via the individual stimulating elements 132 of the stimulation module 130). The function and operation of the stimulation signals r₁-rₘ can be generally similar to the function and operation of the stimulation signals s₁-sₘ described above with reference to FIG. 2. For example, in some embodiments, the signals r₁-rₘ are operable to stimulate the stimulation region 134 simultaneously or nearly simultaneously, while in other embodiments the signals r₁-rₘ are operable to stimulate the stimulation region 134 over a varying time period. Likewise, the (input) augmentation signals a₁-aₙ can be directly mapped to the (output) stimulation signals r₁-rₘ such that there is a simple one-to-one mapping between the signals (e.g., n=m), or the mapping between the augmentation signals a₁-aₙ and stimulation signals r₁-rₘ may not be one-to-one (e.g., n≠m) and may involve complex processing using the adaptive neural model.

Stimulation of the implicated region of the brain produces a measureable output that is either internal or external to the brain, as described above with reference to FIG. 2. For example, the measureable output can be a specific neural activity pattern detected in the nervous system of the subject, a behavioral or motor response of the subject, and/or a sensory response of the subject. At block 408, the process receives an error signal based, at least in part, on this measurable output. For example, as illustrated in FIG. 3, the system 100 can receive either or both of an external error signal ER_{E} and an internal error signal ER₁ from, for example, the error signal generating system 150 (FIG. 1). As described above with reference to FIG. 2, the error signal ER_{E} can correspond to an external error in sensory or motor behavior, and the error signal ER_{I} can correspond to an internal error (e.g., a difference between neural activities patterns in sensory, motor, or other regions of the brain), or an internal reward-based error (e.g., a reward prediction error) measured in the reward centers of the brain of the subject. In some embodiments, the measurable output from the subject is compared with a desired output for the particular task (e.g., a goal in a new motor skill or a new sensory perception task) to generate the error signal.

In certain embodiments for augmenting neural function, utilizing a reward-based error signal can be particularly advantageous since a subject's desired output may be difficult to know beforehand, and therefore cannot be used as a basis for comparison for generating the error signal. For example, where the augmentation signals a₁-aₙ are received from an ultrasonic sensor in a naturalistic setting, it may be difficult to determine a precise desired output for use in determining the error signal. In such a context, a reward-based error signal (e.g., derived from neurochemical signals such as dopamine inside the brain) provides a tool for adjusting and optimizing the neural model that does not require previous knowledge of or measurement of desired outputs for the subject.

At block 410, the process adjusts the neural model based, at least in part, on the error signal. For example, the external or internal error signal can be used to adapt parameters (e.g., one or more sets of weights or network activations, regression parameters, etc.) of the neural model using an optimization method for minimizing error. However, in other embodiments, the process 400 is an adaptive process for augmenting neural function in which the adjustments to the neural model are based on signals or information different than an error signal. The process 400 then returns to the block 402 and can be repeated again. The process 400 causes the system 100 to co-adapt and jointly optimize task performance with the nervous system to (a) incorporate the artificial system 100X) (i.e., a neural co-processor) within the nervous system's computational processing pathways, and (b) promote neuroplasticity, rewiring, and creation of new neural pathways between populations of neurons, allowing the combined nervous system and computing system to learn a new skill or new sensation, and augment the brain's computational capacities. For example, in the embodiment of FIG. 5, new neural pathways can be induced within the brain 500 (e.g., within or between the region of interest 124, stimulation region 134, and other regions directly or indirectly connected to these regions) as represented by the arrows A and B in FIG. 5.

From the foregoing, it will be appreciated that specific embodiments of the present technology have been described herein for purposes of illustration, but that various modifications may be made without deviating from the scope of the present technology. For example, in particular embodiments, the parameters of the neural model can be adjusted based on user input, data, information, and/or signals other than the error signal, either continuously or at certain offline times. That is, receiving an error signal is not a requirement of the neural co-processor (e.g., the system 100) described above with reference to FIGS. 1-5. Rather the system 100 can be used in the absence of an error signal as an additional processing unit for the nervous system. In such embodiments, the system 100 can implement a fixed or non-error-based adaptive computation for mapping neural and/or other sensor inputs to stimulation patterns.

### EXAMPLES

Several aspects of the present technology are set forth in the following examples.

1. A method for augmenting or restoring neural function and inducing new neural connections in a nervous system of a human subject, the method comprising:
receiving (a) multiple neural signals from the nervous system of the subject via a first sensor implanted in and/or worn externally proximate to a region of interest of the nervous system of the subject and/or (b) multiple external signals via a second sensor or information source that is external to the nervous system of the subject;
generating a stimulation pattern based on (a) the neural signals and/or external signals and (b) a neural model;
outputting the stimulation pattern to a stimulation region of the nervous system to produce a measureable output from the subject;
receiving an error signal based on the measureable output;
adjusting at least one parameter of the neural model based on the error signal; and
generating an adjusted neural model.

2. The method of example 1, further comprising receiving neural signals from a region of interest in the brain of a human patient, wherein an injured region of the nervous system is between the region of interest and the stimulation region, and wherein the method further comprises promoting neuroplasticity between the region of interest and the stimulation region to promote recovery and restoration of lost neural function.

3. The method of example 1 or 2, further comprising receiving both neural and external signals associated with a neural function to be augmented, wherein the stimulation region is a region of the brain of the subject that is implicated in the neural function to be augmented, and wherein the method further comprises co-adapting with and promoting neuroplasticity in the brain or another region of the nervous system of the individual.

4. The method of any one of examples 1-3 wherein the neural signals include at least a first neural signal recorded at a first location in the nervous system and a second neural signal recorded simultaneously at a second location in the nervous system different from the first location.

5. The method of any one of examples 1-4 wherein the measurable output is sensory or motor behavior of the subject, and wherein the error signal is based on a difference between a measured sensory or motor behavior of the subject and a desired sensory or motor behavior of the subject.

6. The method of any one of examples 1-5 wherein the measurable output is a neural activity pattern, and wherein the error signal is based on a difference between the neural activity pattern and a desired neural activity pattern.

7. The method of example 6 wherein the neural activity pattern is in a region of the brain associated with sensory, motor, or other behavior.

8. The method of any one of examples 1-7 wherein the measurable output is a reward signal measured at a reward center of the brain of the subject.

9. The method of any one of examples 1-8 wherein the neural model is at least one of an artificial neural network model, a generalized linear model, a logistic regression, a polynomial regression, a Gaussian process regression, or other machine learning method for function approximation, and wherein adjusting the at least one parameter of the neural model includes adjusting a weight or value of one or more variables defining the neural model.

10. The method of any one of examples 1-9, further comprising:
detecting the neural signals while the subject performs a particular task; and
comparing the measureable output to a desired output for the particular task performed by the subject to generate the error signal.

11. The method of any one of examples 1-10 wherein the neural signals are first neural signals, wherein the external signals are first external signals, wherein the stimulation pattern is a first stimulation pattern, wherein the measurable output is a first measurable output, wherein the error signal is a first error signal, and wherein the adjusted neural model is a first adjusted neural model, and wherein the method further comprises:
receiving (a) multiple second neural signals from the nervous system of the subject via the first sensor and/or (b) multiple second external signals via the second sensor or information source;
generating a second stimulation pattern based on (a) the second neural signals and/or second external signals and (b) the first adjusted neural model;
outputting the second stimulation pattern to the stimulation region of the nervous system to produce a second measureable output;
receiving a second error signal based on the second measureable output; and
adjusting at least one parameter of the first adjusted neural model based on the second error signal to generate a second adjusted neural model.

12. A system for inducing new neural connections and restoring lost neural function in a nervous system of a human patient, the system comprising:
at least one sensor configured to record multiple neural signals at a first region of the nervous system, wherein the sensor is implanted within and/or worn externally by the human patient proximate the first region;
at least one stimulating component configured to receive a stimulation pattern and stimulate a second region of the nervous system based on the stimulation pattern to produce a measurable patient output, wherein the stimulating component is implanted within and/or worn externally by the human patient proximate the second region, and wherein the nervous system includes an injured region between or functionally associated with the first region and second region; and
a computing device communicatively coupled to the at least one sensor and the at least one stimulating component, the computing device having a memory containing computer-executable instructions and a processor for executing the computer-executable instructions contained in the memory, wherein the computer-executable instructions include instructions to—
   receive the multiple neural signals from the sensor;
   generate the stimulation pattern based on (a) the received multiple neural signals and (b) a neural model;
   output the stimulation pattern to the stimulating component; and
   adjust at least one parameter of the neural model based on a received error signal, wherein the error signal is based at least in part on the difference between the measurable output and a desired output.

13. The system of example 12 wherein the computing device is implanted in and/or worn by the human patient.

14. The system of example 12 or 13 wherein the injured region is an injured region of the spinal cord of the human patient, wherein the first region is a motor-intention forming region of the brain of the human patient, wherein the second region is a region of the spinal cord different from the injured region, and wherein the system is configured to promote neural plasticity between the first and second regions.

15. The system of any one of examples 12-14 wherein the injured region is an injured region of the brain of the human patient caused by a stroke, traumatic brain injury, or disease, wherein the first region and the second region are regions of the brain disconnected by the injured region, and wherein the system is configured to promote neural plasticity between the first and second regions.

16. The system of any one of examples 12-15 wherein the measureable patient output is external to the nervous system.

17. The system of any one of examples 12-16 wherein the measureable patient output is internal to the nervous system.

18. A system for augmenting a neural function of a nervous system of a human subject, the system comprising:
a first sensor and/or information source configured to receive multiple signals related to the neural function to be augmented;
a second sensor configured to record multiple neural signals from a region of the nervous system related to the neural function to be augmented;
a stimulating component configured to receive a stimulation pattern and stimulate an augmentation region of the nervous system based on the stimulation pattern to produce a measurable output, wherein the stimulating component is implanted in and/or worn by the subject proximate the augmentation region, and wherein the augmentation region is implicated in the neural function to be augmented; and
a computing device communicatively coupled to the first sensor and/or information source, the second sensor, and the stimulating component, the computing device having a memory containing computer-executable instructions and a processor for executing the computer-executable instructions contained in the memory, wherein the computer-executable instructions include instructions to—
   receive (a) the multiple signals from the first sensor and/or information source and (b) the multiple neural signals from the second sensor;
   generate the stimulation pattern based on (a) the received multiple signals, (b) the received multiple neural signals, and (c) a neural model;
   output the stimulation pattern to the stimulating component; and
   adjust at least one parameter of the neural model based on a received error signal, the error signal based at least in part on the difference between the measurable output and a desired output.

19. The system of example 18 wherein the information source is the Internet or another network, and wherein the computing device is configured for wireless communication with the information source.

20. The system of example 18 or 19 wherein the region of the nervous system related to the neural function to be augmented is the prefrontal cortex of the brain of the subject, wherein the augmentation region is a motor region of the brain, and wherein the computing device is configured to augment and/or accelerate the ability of the subject to store a short-term memory, plan, or learn a motor skill.

21. The system of any one of examples 18-20 wherein the first sensor, the second sensor, and the computing device are implanted in and/or worn by the human subject.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. Moreover, the various embodiments described herein may also be combined to provide further embodiments (e.g., the disclosed system may include components for simultaneous augmentation and restoration of function in a nervous system of a subject).

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms may also include the plural or singular term, respectively. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method for augmenting or restoring neural function and inducing new neural connections in a nervous system of a human subject, the method comprising:
receiving (a) multiple neural signals from the nervous system of the subject via a first sensor implanted in and/or worn externally proximate to a region of interest of the nervous system of the subject and/or (b) multiple external signals via a second sensor or information source that is external to the nervous system of the subject;
generating a stimulation pattern based on (a) the neural signals and/or external signals and (b) a neural model;
outputting the stimulation pattern to a stimulation region of the nervous system to produce a measureable output from the subject;
receiving an error signal based on the measureable output;
adjusting at least one parameter of the neural model based on the error signal, and
generating an adjusted neural model.

2. The method of claim 1, further comprising receiving neural signals from a region of interest in the brain of a human patient, wherein an injured region of the nervous system is between the region of interest and the stimulation region, and wherein the method further comprises promoting neuroplasticity between the region of interest and the stimulation region to promote recovery and restoration of lost neural function.

3. The method of claim 1, further comprising receiving both neural and external signals associated with a neural function to be augmented, wherein the stimulation region is a region of the brain of the subject that is implicated in the neural function to be augmented, and wherein the method further comprises co-adapting with and promoting neuroplasticity in the brain or another region of the nervous system of the individual.

4. The method of claim 1 wherein the neural signals include at least a first neural signal recorded at a first location in the nervous system and a second neural signal recorded simultaneously at a second location in the nervous system different from the first location.

5. The method of claim 1 wherein the measurable output is sensory or motor behavior of the subject, and wherein the error signal is based on a difference between a measured sensory or motor behavior of the subject and a desired sensory or motor behavior of the subject.

6. The method of claim 1 wherein the measurable output is a neural activity pattern, and wherein the error signal is based on a difference between the neural activity pattern and a desired neural activity pattern.

7. The method of claim 6 wherein the neural activity pattern is in a region of the brain associated with sensory, motor, or other behavior.

8. The method of claim 1 wherein the measurable output is a reward signal measured at a reward center of the brain of the subject.

9. The method of claim 1 wherein the neural model is at least one of an artificial neural network model, a generalized linear model, a logistic regression, a polynomial regression, a Gaussian process regression, or other machine learning method for function approximation, and wherein adjusting the at least one parameter of the neural model includes adjusting a weight or value of one or more variables defining the neural model.

10. The method of claim 1, further comprising:
detecting the neural signals while the subject performs a particular task, and
comparing the measureable output to a desired output for the particular task performed by the subject to generate the error signal.

11. The method of claim 1 wherein the neural signals are first neural signals, wherein the external signals are first external signals, wherein the stimulation pattern is a first stimulation pattern, wherein the measurable output is a first measurable output, wherein the error signal is a first error signal, and wherein the adjusted neural model is a first adjusted neural model, and wherein the method further comprises:
receiving (a) multiple second neural signals from the nervous system of the subject via the first sensor and/or (b) multiple second external signals via the second sensor or information source;
generating a second stimulation pattern based on (a) the second neural signals and/or second external signals and (b) the first adjusted neural model;
outputting the second stimulation pattern to the stimulation region of the nervous system to produce a second measureable output;
receiving a second error signal based on the second measureable output; and
adjusting at least one parameter of the first adjusted neural model based on the second error signal to generate a second adjusted neural model.

12. A system for inducing new neural connections and restoring lost neural function in a nervous system of a human patient, the system comprising:
at least one sensor configured to record multiple neural signals at a first region of the nervous system, wherein the sensor is implanted within and/or worn externally by the human patient proximate the first region;
at least one stimulating component configured to receive a stimulation pattern and stimulate a second region of the nervous system based on the stimulation pattern to produce a measurable patient output, wherein the stimulating component is implanted within and/or worn externally by the human patient proximate the second region, and wherein the nervous system includes an injured region between or functionally associated with the first region and second region; and
a computing device communicatively coupled to the at least one sensor and the at least one stimulating component, the computing device having a memory containing computer-executable instructions and a processor for executing the computer-executable instructions contained in the memory, wherein the computer-executable instructions include instructions to—
receive the multiple neural signals from the sensor;
generate the stimulation pattern based on (a) the received multiple neural signals and (b) a neural model;
output the stimulation pattern to the stimulating component; and
adjust at least one parameter of the neural model based on a received error signal, wherein the error signal is based at least in part on the difference between the measurable output and a desired output.

13. The system of claim 12 wherein the computing device is implanted in and/or worn by the human patient.

14. The system of claim 12 wherein the injured region is an injured region of the spinal cord of the human patient, wherein the first region is a motor-intention forming region of the brain of the human patient, wherein the second region is a region of the spinal cord different from the injured region, and wherein the system is configured to promote neural plasticity between the first and second regions.

15. The system of claim 12 wherein the injured region is an injured region of the brain of the human patient caused by a stroke, traumatic brain injury, or disease, wherein the first region and the second region are regions of the brain disconnected by the injured region, and wherein the system is configured to promote neural plasticity between the first and second regions.

16. The system of claim 12 wherein the measureable patient output is external to the nervous system.

17. The system of claim 12 wherein the measureable patient output is internal to the nervous system.

18. A system for augmenting a neural function of a nervous system of a human subject, the system comprising:
a first sensor and/or information source configured to receive multiple signals related to the neural function to be augmented;
a second sensor configured to record multiple neural signals from a region of the nervous system related to the neural function to be augmented;
a stimulating component configured to receive a stimulation pattern and stimulate an augmentation region of the nervous system based on the stimulation pattern to produce a measurable output, wherein the stimulating component is implanted in and/or worn by the subject proximate the augmentation region, and wherein the augmentation region is implicated in the neural function to be augmented; and
a computing device communicatively coupled to the first sensor and/or information source, the second sensor, and the stimulating component, the computing device having a memory containing computer-executable instructions and a processor for executing the computer-executable instructions contained in the memory, wherein the computer-executable instructions include instructions to—
receive (a) the multiple signals from the first sensor and/or information source and (b) the multiple neural signals from the second sensor;
generate the stimulation pattern based on (a) the received multiple signals, (b) the received multiple neural signals, and (c) a neural model;
output the stimulation pattern to the stimulating component; and
adjust at least one parameter of the neural model based on a received error signal, the error signal based at least in part on the difference between the measurable output and a desired output.

19. The system of claim 18 wherein the information source is the Internet or another network, and wherein the computing device is configured for wireless communication with the information source.

20. The system of claim 18 wherein the region of the nervous system related to the neural function to be augmented is the prefrontal cortex of the brain of the subject, wherein the augmentation region is a motor region of the brain, and wherein the computing device is configured to augment and/or accelerate the ability of the subject to store a short-term memory, plan, or learn a motor skill.

21. The system of claim 18 wherein the first sensor, the second sensor, and the computing device are implanted in and/or worn by the human subject.
